(19) United States
(12) Patent Application Publication
(10) Pub. No.: US 2005/0226923 A1
(43) Pub. Date: Oct. 13, 2005

(54) Venlafaxine compositions in the form of microtablets

(76) Inventor: Chad Michael Gassert, Hoboken, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/819,879

(22) Filed: Apr. 7, 2004

(51) Int. Cl. A61K 31/275; A61K 9/20

(52) U.S. Cl. 424/464; 514/520

(57) ABSTRACT

A pharmaceutical composition comprising microtablets, wherein said microtablets comprise from about 0.1 weight percent to about 99.9 weight percent of venlafaxine, about 0.1 weight percent to about 20 weight percent of a lubricant, and optionally one or more excipients, wherein said weight percents are based on the total weight of the microtablet, and said microtablets have a tablet size of about 1 mm to about 4 mm, and a tablet weight of 1 to 50 mg. The microtablets of the invention prevent or minimize leaching of the highly water-soluble venlafaxine during an aqueous coating process, and provide a more uniform release rate, as compared to venlafaxine compositions in the form of spheroids or pellets.

### FIELD OF THE INVENTION

The present invention provides a pharmaceutical composition comprising microtablets, wherein said microtablets comprise venlafaxine, a lubricant, and optionally one or more excipients.

### BACKGROUND OF THE INVENTION

Venlafaxine is the non-proprietary name for 1-[2-(dimethylamino)-1-(4-methoxyphenyl)ethyl]cyclohexanol or N,N-dimethyl-2-(1 -hydroxycyclohexyl)-2-(4-methoxyphenyl)ethylamine, and is useful in treating a number of disorders including depression, anxiety, panic disorder and pain. Venlafaxine is administered as venlafaxine hydrochloride in treating depression. See The Merck Index, 12^{th} Edition, entry 10079. Venlafaxine hydrochloride is available as an immediate-release tablet and an extended-release capsule, under the trademark EFFEXOR® and EFFEXOR XR®, respectively, from Wyeth-Ayerst.

U.S. Pat. No. 4,535,186 describes a class of hydroxycycloalkanephenethyl amines as being useful antidepressants and exemplifies the compound now known as venlafaxine hydrochloride as one of the suitable species.

U.S. Pat. No. 6,274,171 and related EP 0 797 991 A1 describe encapsulated extended release formulations of venlafaxine hydrochloride which comprise a hard gelatin capsule containing spheroids comprised of venlafaxine hydrochloride, microcrystalline cellulose and hydroxypropylmethylcellulose coated with ethyl cellulose and hydroxypropylmethylcellulose.

WO 99/22724 describes encapsulated venlafaxine hydrochloride extended-release dosage forms. These formulations differ from those in U.S. Pat. No. 6,274,171 and EP 0 797 991 in that the spheroid is substantially free of HPMC.

U.S. Pat. No. 6,197,828 and WO 00/32556 describe the use of individual (+) and (−) enantiomers of venlafaxine, as well as metabolites thereof. While the commercial venlafaxine hydrochloride is a racemate, these patents teach that various side effects may be reduced by using one isomer substantially without the presence of the other.

Although venlafaxine compositions in the form of spheroids or pellets provides good pharmaceutical activity, it would be beneficial to find other forms for venlafaxine compositions. In particular, venlafaxine compositions that do not require an organic solvent based system for applying an extended release polymer in order to prevent leaching of the highly water-soluble venlafaxine.

### SUMMARY OF THE INVENTION

The invention provides a pharmaceutical composition comprising microtablets, wherein said microtablets comprise from about 0.1 weight percent (wt. %) to about 99.9 wt. % of venlafaxine, about 0.1 wt. % to about 20 wt. % of a lubricant, and optionally one or more excipients, wherein said weight percents are based on the total weight of the microtablet, and said microtablets have a tablet size of about 1 mm to about 4 mm, and a tablet weight of 1 to 50 mg. Preferably, the microtablets are essentially free of a wax.

According to another aspect, the invention provides a dry blending method for preparing a pharmaceutical composition comprising microtablets, wherein said microtablets comprise from about 0.1 wt. % to about 99.9 wt. % of venlafaxine, about 0.1 wt. % to about 20 wt. % of a lubricant, and optionally one or more excipients, wherein said weight percents are based on the total weight of the microtablet, said method comprising: (i) mixing venlafaxine, a lubricant and optionally one or more excipients to form a blend; and (ii) compressing the blend to form a microtablet, wherein said microtablets have a tablet size of about 1 mm to about 4 mm, and a tablet weight of 1 to 50 mg.

According to another aspect, the invention provides a method for treating a disorder which is selected from the group consisting of depression, obesity, panic disorder, post-traumatic stress disorder, attention deficit disorder, Gilles de la Tourette syndrome, bulimia nervosa, anxiety and Shy Drager Syndrome, said method comprising administering to a mammal in need of such treatment, a therapeutically effective amount of a pharmaceutical composition comprising microtablets, wherein said microtablets comprise from about 0.1 wt. % to about 99.9 wt. % of venlafaxine, about 0.1 wt. % to about 20 wt. % of a lubricant and optionally one or more excipients, wherein said weight percents are based on the total weight of the microtablet, and said microtablets have a tablet size of about 1 mm to about 4 mm, and a tablet weight of 1 to 50 mg.

The present inventor has unexpectedly determined that the microtablets of the invention prevent or minimize leaching of the highly water-soluble venlafaxine during an aqueous coating process of an extended release polymer, and provide a more uniform release rate, as compared to venlafaxine compositions in the form of spheroids or pellets. In addition, the microtablets do not require a wax coating.

### DESCRIPTION OF THE INVENTION

The invention provides a pharmaceutical composition comprising microtablets, wherein said microtablets comprise venlafaxine, a lubricant and optionally one or more excipients. As used herein, “venlafaxine” is 1-[2-(dimethylamino)-1-(4-methoxyphenyl)ethyl]cyclohexanol and includes the free base and pharmaceutically acceptable salt forms of venlafaxine, the racemate and its individual enantiomers, and venlafaxine analogs, both as racemates and as their individual enantiomers. The venlafaxine may be used in any polymorphic form, e.g., in the forms known as Form I or Form II. Preferably, the venlafaxine is venlafaxine hydrochloride.

The microtablets are cylindrical with a flat or convex upper side and lower side and with a diameter and height which are preferably approximately equal and, independently of one another, preferably have a tablet size of from about 1 mm to about 4 mm, more preferably from about 1.5 mm to about 2.5 mm. The microtablets have a tablet weight of about 1 mg to about 50 mg. Preferably, the microtablets have a tablet weight of about 2 mg to about 10 mg.

The venlafaxine is present in an amount of from about 0.1 (wt. %) to about 99.9 (wt. %), based on the total weight of the microtablet. Preferably, the venlafaxine is present in an amount of from about 30 (wt. %) to about 75 (wt. %), more preferably about 35 (wt. %) to about 45 (wt. %), based on the total weight of the microtablet.

The lubricant is present in an amount of from about 0.1 (wt. %) to about 20 (wt. %), based on the total weight of the microtablet. Preferably, the lubricant is present in an amount of from about 0.2 (wt. %) to about 10 (wt. %), more preferably about 0.5 (wt. %) to about 5 (wt. %), based on the total weight of the microtablet.

The lubricant is preferably selected from calcium stearate, magnesium stearate, sodium stearate, zinc stearate, glyceryl monostearate, glyceryl palmitostearate, hydrogenated castor oil, hydrogenated vegetable oil, light mineral oil, mineral oil, polyethylene glycol, sodium benzoate, sodium lauryl sulfate, sodium stearyl fumarate, stearic acid and talc. More preferably, the lubricant is selected from calcium stearate, magnesium stearate, sodium stearate, zinc stearate, glyceryl monostearate, glyceryl palmitostearate, hydrogenated castor oil, hydrogenated vegetable oil and stearic acid. Most preferably, the lubricant is magnesium stearate.

While not wishing to be bound by any particular theory, the present inventors believe that the lubricant forms a hydrophobic barrier during the tabletting process which in addition to the density of the microtablets prevents or minimizes leaching of the highly water-soluble venlafaxine during an aqueous coating process.

The amount of venlafaxine in a unit dosage form is an amount effective to treat one or more venlafaxine treatable diseases or conditions as is hereafter defined and can be determined by those skilled in the art without undue experimentation. Generally, this amount ranges from 2 to 300 mg. Contemplated doses include amounts of about 37.5, 75 and 150 mg strengths. For clarity, all amounts of venlafaxine are expressed herein in terms of the weight of the free base, as is conventional in the art.

In a preferred embodiment, the microtablets of the invention are essentially free of a wax. As used herein, “essentially free” means that the microtablet contains less than 2 wt. %, more preferably less than 1 wt. %, based on the weight of the microtablet of a wax. Most preferably, the microtablet does not contain a wax. Examples of waxes include stearyl alcohol or cetyl alcohol, carnuba wax, white wax, yellow wax and microcrystalline wax.

It is within the scope of the invention for the microtablets to include one or more pharmaceutically acceptable excipients. Examples of such excipients are binders, diluents, plasticizers, anti-caking agents, fillers, solubilizing agents, disintegrants, surfactants, flavorants, sweeteners, stabilizers, anti-oxidants, anti-adherents, preservatives, glidants and pigments. A combination of excipients may also be used. Such excipients are known to those skilled in the art, and thus, only a limited number will be specifically referenced.

Preferred binders include, but are not limited to, starches, e.g., potato starch, wheat starch and corn starch; gums, such as gum tragacanth, acacia gum and gelatin; and polyvinyl pyrrolidone, e.g., Povidone. Polyvinyl pyrrolidone is a particularly preferred binder.

Preferred fillers include, but are not limited to, microcrystalline cellulose, starch, pregelatinized starch, modified starch, dibasic calcium phosphate dihydrate, calcium sulfate trihydrate, calcium sulfate dihydrate, calcium carbonate, dextrose, sucrose, lactose, mannitol and sorbitol. Lactose is a particularly preferred filler.

Examples of disintegrants include:
(i) natural starches, such as maize starch, potato starch and the like, directly compressible starches, e.g., Sta-rx® 1500; modified starches, e.g., carboxymethyl starches and sodium starch glycolate, available as Primojel®, Explotab®, Explosol®; and starch derivatives, such as amylose;
(ii) cross-linked polyvinylpyrrolidones, e.g., crospovidones, such as Polyplasdone® XL and Kollidon® CL;
(iii) alginic acid and sodium alginate;
(iv) methacrylic acid-divinylbenzene co-polymer salts, e.g., Amberlite® IRP-88; and
(v) cross-linked sodium carboxymethylcellulose, available as, e.g., Ac-di-sol®, Primellose®, Pharmacel® XL, Explocel® and Nymcel® ZSX.

Additional disintegrants also include hydroxypropyl cellulose, hydroxypropylmethyl cellulose, croscarmellose sodium, sodium starch glycolate, polacrillin potassium, polyacrylates, such as Carbopol®, magnesium aluminium silicate and bentonite.

Examples of surfactants include:
(i) Reaction products of a natural or hydrogenated castor oil and ethylene oxide. The polyethyleneglycol-hydrogenated castor oils available under the trademark CREMOPHOR are especially suitable, such as CREMOPHOR RH 40 and CREMOPHOR RH 60. Also suitable are polyethyleneglycol castor oils, such as that available under the trade name CREMOPHOR EL.
(ii) Polyoxyethylene-sorbitan-fatty acid esters, also called polysorbates, e.g., mono- and tri-lauryl, palmityl, stearyl and oleyl esters of the type known and commercially-available under the trademark TWEEN.
   20 [polyoxyethylene(20)sorbitanmonolaurate],
   21 [polyoxyethylene(4)sorbitanmonolaurate],
   40 [polyoxyethylene(20)sorbitanmonopalmitate],
   60 [polyoxyethylene(20)sorbitanmonostearate],
   65 [polyoxyethylene(20)sorbitantristearate],
   80 [polyoxyethylene(20)sorbitanmonooleate],
   81 [polyoxyethylene(5)sorbitanmonooleate],
   85 [polyoxyethylene(20)sorbitantrioleate].

A preferred product of this class is TWEEN 80.

Although polyethylene glycol (PEG) itself does not function as a surfactant, a variety of PEG-fatty acid esters have useful surfactant properties. Among the PEG-fatty acid monoesters, esters of lauric acid, oleic acid and stearic acid are most useful. Among the surfactants of Table 1, preferred hydrophilic surfactants include PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate.
(iii) Polyoxyethylene fatty acid esters, for example polyoxyethylene stearic acid esters of the type known and commercially available under the trademark MYRJ.
(iv) Polyoxyethylene-polyoxypropylene co-polymers and block co-polymers, e.g., of the type known and commercially-available under the trademark PLURONIC, EMKALYX and POLOXAMER. Preferred products of this class are PLURONIC F68 and POLOXAMER 188.
(v) Dioctylsulfosuccinate or di-[2-ethylhexyl]-succinate.
(vi) Phospholipids, in particular, lecithins. Suitable lecithins include, in particular, soybean lecithins.
(vii) Propylene glycol mono- and di-fatty acid esters, such as propylene glycol dicaprylate (also known and commercially-available under the trademark MIGLYOL 840), propylene glycol dilaurate, propylene glycol hydroxystearate, propylene glycol isostearate, propylene glycol laurate, propylene glycol ricinoleate and propylene glycol stearate.
(viii) Polyoxyethylene alkyl ethers, such as those commercially-available under the trademark BRIJ, e.g., BRIJ 92V and BRIJ 35.
(ix) Tocopherol esters, e.g., tocopheryl acetate and tocopheryl acid succinate.
(x) Docusate salts, e.g., dioctylsulfosuccinate or related compounds, such as di-[2-ethylhexyl]-succinate.

A combination of surfactants may also be used.

Preferred sweeteners include, but are not limited to, artificial sweeteners, such as aspartame, saccharin and cyclamates; natural sweeteners, such as sucrose, fructose, glucose, lactose, maltodextrin and sodium glycolate; and mixtures of artificial and natural sweeteners, such as a mixture of aspartame and sucrose.

Preferred pigments include, but are not limited to, titanium dioxide, iron oxide and vegetable dyes.

Preferred diluents include, but are not limited to, dextrose, sorbitol, sucrose, lactose, mannitol, urea, potassium chloride, sodium chloride, gelatin, starch, methyl cellulose, ethyl cellulose, propyl cellulose, hydroxymethyl cellulose, hydroxyethyl cellulose, hydroxypropyl cellulose, hydroxypropyl methyl cellulose, silica, polyvinyl alcohol, polyvinylpyrrolidone and magnesium stearate.

The microtablets may be coated or uncoated. Preferred coatings include the following: cross-linked polyvinyl pyrrolidone, non-cross linked polyvinylpyrrolidone, hydroxypropylmethyl cellulose phthalate, hydroxypropylmethyl cellulose acetate succinate, cellulose acetate succinate, cellulose acetate phthalate, hydroxypropylmethyl cellulose acetate succinate, cellulose acetate trimellitate; hydroxypropyl methyl cellulose phthalate, hydroxypropyl methyl cellulose acetate succinate, starch acetate phthalate, polyvinyl acetate phthalate, carboxymethyl cellulose, methyl cellulose phthalate, methyl cellulose succinate, methyl cellulose phthalate succinate, methyl cellulose phthalic acid half ester, ethyl cellulose succinate, carboxymethylamide, potassium methacrylatedivinylbenzene co-polymer, polyvinylalcohols, co-polymers of acrylic acid and/or methacrylic acid with a monomer selected from the following: methyl methacrylate, ethyl methacrylate, ethyl acrylate, butyl methacrylate, hexyl methacrylate, decyl methacrylate, lauryl methacrylate, phenyl methacrylate, methyl acrylate, isopropyl acrylate, isobutyl acrylate, or octadecyl acrylate, e.g., EUDRAGIT®-L, -S, and -NE series, such as L100-55, L30D55, L100, S100, L12.5, S12.5, and NE 30D, available from Rohm; polyvinyl acetate; fats; oils; waxes; fatty alcohols; shellac; gluten; ethylacrylate-maleic acid anhydride co-polymer; maleic acid anhydride-vinyl methyl ether co-polymer; styrol-maleic acid co-polymer; 2-ethyl-hexyl-acrylate maleic acid anhydride; crotonic acid-vinyl acetate co-polymer; glutaminic acid/glutamic acid ester co-polymer; carboxymethylethylcellulose glycerol monooctanoate; polyarginine; poly(ethylene); poly(propylene); poly(ethylene oxide); poly(ethylene terephthalate); poly(vinyl isobutyl ether); poly(vinyl chloride); and polyurethane. A combination of coatings may also be used.

More preferably, the coating is a methacrylic ester co-polymer (EUDRAGIT® NE 30D).

The coating is preferably present in an amount of from about 2 wt. % to about 25 wt. %, based on the total weight of the microtablet. More preferably, the coating is present in an amount of from about 5 wt. % to about 15 wt. %, based on the total weight of the microtablet. The coating is applied to the microtablets by conventional coating techniques, such as coating in a tank or a fluidized bed employing polymer solutions in water or in suitable organic solvents or using latex suspensions of these polymers.

The coating component of the invention may contain a plasticizer. The amount of plasticizer is in general optimized for each coating polymer and generally represents from about 1 wt. % to about 50 wt. %, preferably 2 to 20 wt. %, based on the total weight of the coating polymer.

Preferred plasticizers include, but are not limited to, citric and tartaric acid esters, (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates, such as Polysorbar 50); sorbitan monooleate. A combination of plasticizers may also be used.

The microtablets of the invention are preferably prepared by mixing venlafaxine and optionally one or more excipients, in the presence or absence of a solvent, to form a blend. The blend is preferably in the form of a solid dispersion or a homogeneous suspension. The blend is preferably dried, and optionally milled and/or screened. Useful drying techniques include spray drying, fluid bed drying, flash drying, ring drying, tray drying, vacuum drying, radio-frequency drying and microwave drying. A preferred drying technique is fluid bed. Useful mills include fluid energy mill, ball mill or rod mill, hammer mill, cutting mill and oscillating granulator. More specifically, suitable mills include, Quadro Comill, Fryma, Glatt Quick Sieve, Fluidaire, Fitzpatrick (Fitz mill), BTS mill and Tornado. A preferred mill is a Fitz mill.

The blend is preferably subject to compression, wet granulation or roller compaction, to form microtablets. The tabletting takes place in a suitable tabletting machine equipped with multiple microtablet punches.

In one embodiment of the invention, the microtablets are enclosed inside a capsule, e.g., a gelatin capsule. For this, any gelatin capsule conventionally employed in the pharmaceutical formulation field can be used, such as the hard gelatin capsule or a HPMC capsule, such as Vegicap®.

The microtablets of the invention are particularly suitable for oral administration of venlafaxine and are intended to include all methods of treating depression, obesity, panic disorder, post-traumatic stress disorder, late luteal phase dysphoric disorder (premenstrual syndrome), attention deficit disorders, with and without hyperactivity, Gilles de la Tourette syndrome, bulimia nervosa, generalized anxiety disorder or Shy Drager Syndrome in mammals, preferably in humans.

The following non-limiting examples illustrate further aspects of the invention.

### EXAMPLE 1

Preparation of Venlafaxine Extended-Release Microtablets

| | |
|---|---|
| % | Mg/unit |

TABLET CORE

| | | |
|---|---|---|
| Veniafaxine HCI | 40 | 1.680 |
| Lactose, sprayed dried | 38.3 | 1.610 |
| Vinylpyrrolidone/Vinylacetate | 20 | 0.840 |
| co-polymer (Copovidone) | | |
| Silicon dioxide, fumed | 0.2 | 0.008 |
| Magnesium stearate | 1.5 | 0.063 |
| Total | 100 | 4.2 |

TABLET COATING

| | | | |
|---|---|---|---|
| EUDRAGIT ®NE 30D (30% | 11 | 0.462 | 1.54 |
| dispersion of methacrylic ester | | (based on | |
| copolymer in water) | | polymer) | |
| Talc | 4.7 | 0.231 | |
| Deionizedwater | 115.7 | 4.893 | |
| Total | | | |

The microtablet core was prepared by blending venlafaxine, lactose, copovidone, and silicone dioxide in a V-blender for 5 minutes at 25 rpm, to form a mixture. Magnesium stearate added to the mixture and blended for 1.5 minutes at 25 rpm to form a homogeneous blend. The blend was compressed on a Korsch XL 100 equipped with microtablet tooling. Tooling is nine 2 mm diameter carbide tips per punch in a circular configuration to yield microtablets having a diameter of 2 mm and a height of approximately 1.5 mm.

The coating was prepared by dispersing EUDRAGIT® NE 30D in water and adding talc with mixing. The coating was deposited onto the microtablets using a Vector LDCS partially perforated coating pan. The coated tablets were encapsulated using an H&K Encapsulator equipped with a pellet feeder.

### EXAMPLE 2

The microtablets prepared in Example 1 were packed into hard gelatin capsules using a suitable capsule filling machine. Individually, three capsules containing approximately 49 of the microtablets from Example 1 were prepared and designated Sandoz Capsule 1, Sandoz Capsule 2 and Sandoz Capsule 3. Each capsule contained 75 mg of venlafaxine hydrochloride. Separately, three capsules of EFFEXOR XR® which contained spheroids packed into a hard gelatin capsule containing 75 mg of venlafaxine hydrochloride, cellulose, ethyl cellulose, gelatin, hydroxypropyl methylcellulose, iron oxide and titanium dioxide, were designated Effexor Capsule 1, Effexor Capsule 2 and Effexor Capsule 3.

The six capsules were evaluated in a dissolution study. Each capsule was placed in a basket of a dissolution apparatus containing 900 mL deionized water, and the basket was rotated at 100 rpm for 24 hours. Samples were taken every hour and the amount of venlafaxine hydrochloride was determined by UV. The dissolution profile for the Sandoz capsules are summarized in Table 1. The dissolution profile for the EFFEXOR XR® capsules are summarized in Table 2.

TABLE 2
Dissolution Study for Sandoz Capsules

| | Sandoz | Sandoz | Sandoz | |
|---|---|---|---|---|
| Time (hours) | Capsule 1 | Capsule 2 | Capsule 3 | Mean |
| 0 | 0 | 0 | 0 | 0 |
| 1 | 0.5 | −0.1 | −3.4 | −1.0 |
| 2 | 3.7 | 2.4 | 1.6 | 2.6 |
| 3 | 10.4 | 8.1 | 6.9 | 8.5 |
| 4 | 20.7 | 19 | 17.9 | 19.2 |
| 5 | 30.4 | 28.4 | 27.4 | 28.7 |
| 6 | 37.1 | 35.1 | 34.3 | 35.5 |
| 7 | 43.3 | 41.1 | 40.4 | 41.6 |
| 8 | 48.7 | 46.4 | 45.7 | 46.9 |
| 9 | 54.1 | 51.7 | 50.9 | 52.2 |
| 10 | 59.4 | 56.6 | 55.8 | 57.3 |
| 11 | 63.4 | 60.6 | 60.2 | 61.4 |
| 12 | 67.1 | 64 | 63.8 | 65 |
| 13 | 70.5 | 67.4 | 67.2 | 68.4 |
| 14 | 74 | 70.9 | 70.8 | 71.9 |
| 15 | 77.2 | 73.9 | 73.8 | 75 |
| 16 | 80.1 | 76.5 | 76.5 | 77.7 |
| 17 | 82.8 | 79.1 | 79.1 | 80.3 |
| 18 | 85.4 | 81.5 | 81.5 | 82.8 |
| 19 | 88.1 | 84 | 83.8 | 85.3 |
| 20 | 91 | 86.2 | 86 | 87.7 |
| 21 | 93.9 | 88.6 | 88.2 | 90.2 |
| 22 | 96.6 | 91.1 | 90.5 | 92.7 |
| 23 | 99.3 | 93.7 | 92.9 | 95.3 |
| 24 | 101.6 | 96.2 | 95 | 97.6 |

Dissolution Study for EFFEXOR XR ®Capsules

| | Effexor | Effexor | Effexor | |
|---|---|---|---|---|
| Time (hours) | Capsule 1 | Capsule 2 | Capsule 3 | Mean |
| 0 | 0 | 0 | 0 | 0 |
| 1 | 2.8 | 2.9 | 3.1 | 2.9 |
| 2 | 14.4 | 14.2 | 15.7 | 14.8 |
| 3 | 27.5 | 27.4 | 29.2 | 28.0 |
| 4 | 39.3 | 38.6 | 40.9 | 39.6 |
| 5 | 48.7 | 48.2 | 50.1 | 49.0 |
| 6 | 56.2 | 55.6 | 57.4 | 56.4 |
| 7 | 62.6 | 61.8 | 63.8 | 62.7 |
| 8 | 67.9 | 67.1 | 69.1 | 68.0 |
| 9 | 72.6 | 71.8 | 73.8 | 72.7 |
| 10 | 76.3 | 75.7 | 77.8 | 76.6 |
| 11 | 79.2 | 78.7 | 80.9 | 79.6 |
| 12 | 81.7 | 81.1 | 83.6 | 82.1 |
| 13 | 83.7 | 83.4 | 85.9 | 84.3 |
| 14 | 85.8 | 85.9 | 88.6 | 86.8 |
| 15 | 87.5 | 88 | 90.8 | 88.8 |
| 16 | 88.9 | 90 | 92.8 | 90.6 |
| 17 | 90.3 | 91.9 | 94.7 | 92.3 |
| 18 | 91.5 | 93.8 | 96.6 | 94.0 |
| 19 | 92.7 | 95.6 | 98.4 | 95.6 |
| 20 | 94.3 | 97.8 | 100.5 | 97.5 |
| 21 | 96 | 100.1 | 102.6 | 99.6 |
| 22 | 97.8 | 102.4 | 104.4 | 101.5 |
| 23 | 99.4 | 104.5 | 106.2 | 103.4 |
| 24 | 100.9 | 106.7 | 108.1 | 105.2 |

The results in Tables 1 and 2 clearly show that the Sandoz capsules containing the microtablets prepared according to the invention display a uniform dissolution or release rate of venlafaxine hydrochloride which is reproducible as determined by the evaluation of three capsules containing the microtablets of the invention. In addition, the results show that the dissolution rate of the Sandoz capsules are comparable to the dissolution rate of the commercially available EFFEXOR XR® capsules over a period of 24 hours.

### EXAMPLE 3 (COMPARATIVE)

Preparation of Venlafaxine Hydrochloride Pellets

Venlafaxine HCl pellets are prepared by mixing venlafaxine hydrochloride, microcrystalline cellulose, hydroxypropylmethyl cellulose, and water. The mixture is extruded, spheronized and dried to provide uncoated pellets containing 47.27 weight percent of venlafaxine hydrochloride, 52.21 weight percent of microcrystalline cellulose, and 0.52 weight percent of hydroxypropylmethyl cellulose, wherein the weight percents are based on the total weight of the pellet.

Five samples, Ex. 3(a)-Ex. 3(e), are prepared which contain the above described pellets having different coatings, as follows:
Ex. 3(a) is coated with 14.5% by weight of polymer of Aquacoat® CPD which is a 30% by weight dispersion of cellulose acetate polymer in water, and is available from FMC.
Ex. 3(b) is coated with 21.5% by weight of polymer of Aquacoat® CPD.
Ex. 3(c) is coated with 14.33% by weight of polymer of Surelease® which is an aqueous ethylcellulose dispersion, and is available from Colorcon.
Ex. 3(d) is first coated with 5 % by weight cetyl stearyl alcohol and then coated with 12% by weight of polymer of Surelease®.
Ex. 3(e) is first coated with 2.5 % by weight cetyl stearyl alcohol and then coated with 15% by weight of polymer of Eudragit® NE 30D which is a 30% by weight dispersion of methacrylic ester copolymer in water, and is available from Rohm.

The coated pellets are packed into a hard gelatin capsule using a suitable capsule filling machine. The five samples are compared to EFFEXOR XR® capsules wherein the pellets are coated with 25% by weight of ethyl cellulose, and 6.75% by weight of hydroxypropylmethylcellulose 2910, in a 1:1 v/v mixture of methylene chloride and anhydrous methanol.

The six capsules are evaluated in a dissolution study. Each capsule is placed in a basket of a dissolution apparatus containing 900 mL deionized water, and the basket was rotated at 100 rpm for 24 hours. Samples were taken every hour and the amount of venlafaxine hydrochloride was determined by UV. The dissolution profile for each of the capsules is summarized in Table 3.

TABLE 3
Dissolution Profile for Pellets Having Different Coatings

| | | | | | | Effexor |
|---|---|---|---|---|---|---|
| Time | Ex. 3(a) | Ex. 3(b) | Ex. 3(c) | Ex. 3(d) | Ex. 3(e) | Capsule |
| 2 hours | 100 | 84 | 100 | 09 | 08 | 14 |
| 4 hours | | | | 15 | 32 | 40 |
| 8 hours | | | | 19 | 68 | 67 |
| 12 hours | | | | | 85 | 79 |
| 24 hours | | | | 35 | 98 | 93 |

The results in Table 3 clearly show that the pellets coated with different types and amounts of aqueous based coatings without the addition of a wax do not provide an extended release over a 24 hour period of the venlafaxine hydrochloride. Only Ex. 3(d) and Ex. 3(e) which were coated with cetyl stearyl alcohol exhibited an extended release profile. The commercially available EFFEXOR XR® capsule exhibited an extended release profile, however, an organic solvent was necessary to apply the extended release polymer.

The microtablets of the invention prevent or minimize leaching of the highly water-soluble venlafaxine during an aqueous coating process of an extended release polymer, and provide a more uniform release rate, as compared to venlafaxine compositions in the form of spheroids or pellets. In addition, the microtablets do not require a wax coating.

While the invention has been described with particular reference to certain embodiments thereof, it will be understood that changes and modifications may be made by those of ordinary skill within the scope and spirit of the following claims:

1. A pharmaceutical composition comprising microtablets, wherein said microtablets comprise from about 0.1 to about 99.9 weight percent of venlafaxine, about 0.1 weight percent to about 20 weight percent of a lubricant, and optionally one or more excipients, wherein said weight percents are based on the total weight of the microtablet, and said microtablets have a tablet size of about 1 mm to about 4 mm, and a tablet weight of 1 to 50 mg.

2. A pharmaceutical composition comprising microtablets, wherein said microtablets comprise from about 0.1 to about 99.9 weight percent of venlafaxine, about 0.1 weight percent to about 20 weight percent of a lubricant, and optionally one or more excipients, wherein said weight percents are based on the total weight of the microtablet, and said microtablets have a tablet size of about 1 mm to about 4 mm, and a tablet weight of 1 to 50 mg, wherein said microtablets are essentially free of a wax.

3. The composition according to claim 1, wherein the lubricant is selected from the group consisting of calcium stearate, magnesium stearate, sodium stearate, zinc stearate, glyceryl monostearate, glyceryl palmitostearate, hydrogenated castor oil, hydrogenated vegetable oil, light mineral oil, mineral oil, polyethylene glycol, sodium benzoate, sodium lauryl sulfate, sodium stearyl fumarate, stearic acid, talc and combinations thereof.

4. The composition according to claim 3, wherein the lubricant is selected from the group consisting of calcium stearate, magnesium stearate, sodium stearate, zinc stearate, glyceryl monostearate, glyceryl palmitostearate, hydrogenated castor oil, hydrogenated vegetable oil and stearic acid.

5. The composition according to claim 4, wherein the lubricant is selected from the group consisting of stearic acid, calcium stearate, magnesium stearate, sodium stearate and zinc stearate.

6. The composition according to claim 5, wherein the lubricant is magnesium stearate.

7. The composition according to claim 1, wherein the lubricant is present in an amount of from about 0.2 weight percent to about 10 weight percent

8. The composition according to claim 7, wherein the lubricant is present in an amount of from about 0.5 weight percent to about 5 weight percent

9. The composition according to claim 1, wherein the microtablets are enclosed within a capsule.

10. The composition according to claim 1, wherein the venlafaxine is present in an amount of from about 30 weight percent to about 75 weight percent, based on the total weight of the microtablet.

11. The composition according to claim 10, wherein the venlafaxine is present in an amount of from about 35 weight percent to about 45 weight percent, based on the total weight of the microtablet.

12. The composition according to claim 1, wherein the microtablets have a tablet size of about 1.5 mm to about 2.5 mm.

13. The composition according to claim 1, wherein the microtablets have a tablet weight of 2 to 10 mg

14. The composition according to claim 1, wherein the excipient is selected from the group consisting of binders, diluents, plasticizers, anti-caking agents, fillers, solubilizing agents, disintegrants, surfactants, enteric coating, flavorants, sweeteners, stabilizers, anti-oxidants, anti-adherents, preservatives, glidants, pigments and combinations thereof.

15. A dry blending method for preparing a pharmaceutical composition comprising micrbtablets, wherein said microtablets comprise from about 0.1 weight percent to about 99.9 weight percent of venlafaxine, about 0.1 weight percent to about 20 weight percent of a lubricant, and optionally one or more excipients, wherein said weight percents are based on the total weight of the microtablet, said method comprising: (i) mixing venlafaxine, a lubricant, and optionally one or more excipients to form a blend; and (ii) compressing the blend to form a microtablet, wherein said microtablets have a tablet size of about 1 mm to about 4 mm, and a tablet weight of 1 to 50 mg.

16. A method for treating depression comprising administering to a mammal in need of such treatment, a therapeutically effective amount of a pharmaceutical composition comprising microtablets, wherein said microtablets comprise from about 0.1 weight percent to about 99.9 weight percent of venlafaxine, about 0.1 weight percent to about 20 weight percent of a lubricant, and optionally one or more excipients, wherein said weight percents are based on the total weight of the microtablet, and said microtablets have a tablet size of about 1 mm to about 4 mm, and a tablet weight of 1 to 50 mg.

17. A method for treating a disorder which is selected from the group consisting of obesity, panic disorder, post-traumatic stress disorder, attention deficit disorder, Gilles de la Tourette syndrome, bulimia nervosa, anxiety and Shy Drager Syndrome, said method comprising administering to a mammal in need of such treatment, a therapeutically effective amount of a pharmaceutical composition comprising microtablets, wherein said microtablets comprise from about 0.1 weight percent to about 99.9 weight percent of venlafaxine, about 0.1 weight percent to about 20 weight percent of a lubricant, and optionally one or more excipients, wherein said weight percents are based on the total weight of the microtablet, and said microtablets have a tablet size of about 1 mm to about 4 mm, and a tablet weight of 1 to50mg.

18. A therapeutic package comprising a container and a pharmaceutical composition comprising microtablets, wherein said microtablets comprise from about 0.1 weight percent to about 99.9 weight percent of venlafaxine, about 0.1 weight percent to about 20 weight percent of a lubricant, and optionally one or more excipients, wherein said weight percents are based on the total weight of the microtablet, and said microtablets have a tablet size of about 1 mm to about 4 mm, and a tablet weight of 1 to 50 mg.
